(19) United States
(12) Patent Application Publication
(10) Pub. No.: US 2003/0073737 A1
(43) Pub. Date: Apr. 17, 2003

(54) Tolerance induction

(76) Inventors: Nigel Graham Cooke, Oberwil (CH); Christoph Heusser, Oberwil (CH)

Correspondence Address: THOMAS HOXIE, NOVARTIS, PATENT AND TRADEMARK DEPARTMENT, ONE HEALTH PLAZA 430/2, EAST HANOVER, NJ, 07936-1080, US

(21) Appl. No.: 10/268,712

(22) Filed: Oct. 10, 2002

(30) Foreign Application Priority Data
Oct. 11, 2001 (GB) 0124545.5

(51) Int. Cl. A61K 31/34

(52) U.S. Cl. 514/473

(57) ABSTRACT

Tautomycetin or a derivative thereof is used to induce tolerance, e.g. unresponsiveness to alloantigens, in a subject.

The present invention relates to a method for inducing tolerance in a subject using a compound group comprising tautomycetin.

Tautomycetin is a known antibiotic produced by e.g. Streptomyces griseochromogenes, having the formula I

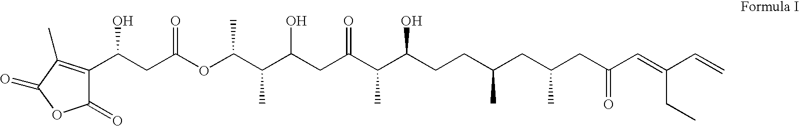

See e.g. Antibiotics, 42, 141-144 (1989) and H. Oikawa et al., Tetrahedron Letters, Vol. 38, No 45, 7897-7900 (1997) for its synthesis.

Tautomycetin has, on the basis of observed activity, e.g. inhibition of the mixed lymphocyte reaction e.g. as described in WO 99/50388, been found to be useful e.g. as an immunosuppressant, e.g. in the treatment of acute allograft rejection.

Over the last two decades, organ transplantation has become a routine therapeutic option for patients with end-stage organ failure. Both short-term and long-term outcomes after organ transplantation have improved; nevertheless, long-term morbidity and mortality still remain substantial problems. The chronic immunosuppressive treatment that organ transplant recipients require for the rest of their lives frequently fails to prevent graft loss due to chronic rejection and is associated with severe side-effects, including infections, malignancies, nephrotoxicity and/or metabolic disorders. The induction of immunological tolerance could provide a solution to these pressing problems not only in the field of allotransplantation but also in certain autoimmune diseases, i.e. autoimmune diseases which are dependent on lymphocyte activation.

It has now been found that tautomycetin or a derivative thereof surprisingly is capable of inducing tolerance, e.g. inducing unresponsiveness to alloantigens in vivo.

By “derivative of tautomycetin” is to be understood a biologically active fragment or derivative thereof, e.g. a compound which exhibits a qualitatively similar effect to that of tautomycetin, or a prodrug thereof, e.g. a physiologically hydrolysable ether or ester thereof.

Tautomycetin or a derivative thereof may exist in free form or in a pharmaceutically acceptable salt form, e.g. a salt obtained by addition of a base, e.g. an alkali or alkali earth metal oxide, for example sodium methoxide or ethoxide or potassium t-butoxide. A tautomycetin derivative may also exist in the form of an acid addition salt.

In accordance with the particular findings of the present invention, there is provided:

1.1 A method for inducing or modulating T or B cell tolerance in a subject in need of such treatment, comprising administering to said subject an effective amount of tautomycetin or a derivative thereof, or a pharmaceutically acceptable salt thereof.

The subjects in need of such treatment may be allotransplanted patients or patients developing autoimmune disorders or diseases which are dependent on activation of lymphoid cells e.g. such as T cells.

Allotransplanted patients may be patients (or recipients) receiving, from a donor, cells, tissues or a solid organ, e.g. pancreatic islet cells, bone marrow, corneal tissue, neuronal tissue, skin, heart, lung, combined heart-lung, kidney, liver, pancreas, bowel, trachea or oesophagus.

1.2 A method for inducing apoptosis of activated T cells in a subject in need of such treatment, comprising administering to said subject an effective amount of tautomycetin or a derivative thereof, or a pharmaceutically acceptable salt thereof.

1.3 A method for inhibiting a rejection response in a recipient subject by inducing immune tolerance in said subject to foreign mammalian donor cells, tissue or organ, comprising the steps of:

a) administering to the recipient an effective amount of tautomycetin or a derivative thereof, or a pharmaceutically acceptable salt thereof;

b) transplanting the donor cells, tissue or organ into the recipient such that a rejection response by the recipient to the donor cells, tissue or organ is inhibited or prevented,

and optionally

c) transplanting bone marrow donor cells or other hemapoietic-precursor-cells or lymphoid donor cells to the recipient such that a graft-versus-host response by the donor cells against the recipient is prevented or inhibited.

Above method may also be performed in any different sequence, e.g. step b), then step a) and optionally step c); or a), then c) and b); or c) then a) followed by b); or c) then b) then a); or all at the same time; or a) and c) alone.

1.4 A method for delaying progression of, attenuating severity of, suppressing, mitigating or treating autoimmune disorders or diseases in a subject by inducing immune tolerance in said subject, the autoimmune disorders or diseases being dependent on activation of lymphoid cells, e.g. such as T cells, which method comprises administering to said subject an effective amount of tautomycetin or a derivative thereof or a pharmaceutically acceptable salt thereof.

Examples of such autoimmune disorders or diseases include e.g. rheumatoid arthritis, multiple sclerosis, systemic lupus erythematosus, asthma, psoriasis, allergic disorders and inflammatory bowel diseases, e.g. Crohn's disease or ulcerative colitis.

1.5 A method for treating malignancies in a subject in need thereof, comprising administering to said subject a therapeutically effective amount of tautomycetin or a derivative thereof or a pharmaceutically acceptable salt thereof.

Examples of malignancies are e.g. hematologic malignancies, for example acute or chronic myeloid leukemias, multiple myelomas, non-Hodgkin's lymphoma, Hodgkin's disease, lymphomas, T-cell leukemias, etc., and other malignancies, e.g. breast cancer.

As alternative to the above the present invention also provides:

2. A pharmaceutical composition for use in any method as defined under 1.1 to 1.5 above comprising tautomycetin or a derivative thereof or a pharmaceutically acceptable salt thereof, together with one or more pharmaceutically acceptable diluents or carriers therefor.

According to the method of the invention, tolerance is induced when e.g. the reactivity or the number of transplant recipient's peripheral blood T-cell reactive against donor cells is reduced by at least 80%, preferably 95% or higher. When the donor organ, tissue or cells are transplanted into the recipient, a rejection response by the recipient to the graft is inhibited and the recipient is tolerized to the donor graft, i.e. the donor graft is accepted as self and further immunosuppressive treatments become no longer necessary, e.g. for a prolonged period of time.

The above described method for inducing tolerance may be complemented by additional treatment regimens; this may be necessary in the context of co-transplantation with donor bone marrow or stem cells. For example, the method can further include partial whole body irradiation and/or local irradiation of the thymus gland before, at the same time, or after the administration of tautomycetin or a derivative thereof.

In accordance with a further embodiment of the invention, systemic or thymic injection of donor leukocytes or lymphocytes having MHC antigen of the same haplotype as the MHC of the donor cell can be administered to the recipient. Thymic injection of a saline solution or a crystalloid or colloid solution to disrupt thymic integrity and increase access of tautomycetin or derivative thereof to the thymus may also be beneficial.

The present tolerance induction method can also include administering an immunosuppressant, immunomodulatory or costimulation blocking compound or a combination of such compounds before, at the same time or after administration of tautomycetin or derivative thereof or a pharmaceutically acceptable salt thereof. Examples of such compounds for adjunction include e.g. a calcineurin inhibitor, e.g. cyclosporin A or FK 506; a mTOR inhibitor e.g. rapamycin or 40-O-(2-hydroxyethyl)-rapamycin, ABT578 or a rapalog as disclosed e.g. in WO 98/02441 or WO 01/14387, e.g. AP23573 or AP22594; cyclophosphamide; azathioprene; methotrexate; leflunomide; mizoribine; mycophenolic acid or a salt thereof, e.g. Myfortic^{R}; mycophenolate mofetil; 15-deoxyspergualine or an immunosuppressive homologue, analogue or derivative thereof; normal or high dose corticosteroid; an accelerating lymphocyte homing agent, e.g. an EDG receptor agonist or a chemokine receptor antagonist, e.g. FTY720 (2-amino-2-[2-(4-octylphenyl) ethyl]propane-1,3-diol in free form or in a pharmaceutically salt form, e.g. the hydrochloride) or Y-36018; immunosuppressive monoclonal antibodies, e.g., monoclonal antibodies to leukocyte receptors, e.g., MHC, CD2, CD3, CD4, CD7, CD8, CD25, CD28, CD40, CD45, CD58, CD80, CD86, CD134, CD137, CD152, ICOS or to their ligands, e.g. CD 154; a recombinant binding molecule having at least a portion of the extracellular domain of CTLA4 or a mutant thereof, e.g. an at least extracellular portion of CTLA4 or a mutant thereof joined to a non-CTLA4 protein sequence, e.g. CTLA4Ig (for ex. designated ATCC 68629) or a mutant thereof, e.g. LEA29Y; adhesion molecule inhibitors, e.g. a LFA-1 antagonist, ICAM-1 or -3 antagonist, VCAM-4 antagonist or VLA-4 antagonist.

Any one, two or more of these adjunct therapies can be used together in the present tolerance induction method. Thus, the invention includes at least 8 methods of inducing tolerance in a subject using tautomycetin or a derivative thereof:

(1) administration to said subject of tautomycetin alone or a derivative thereof or a pharmaceutically acceptable salt thereof;

(2) administration to said subject of tautomycetin or a derivative thereof or a pharmaceutically acceptable salt thereof, associated to other means that alter T cell regulation and/or thymic induction, e.g. as indicated above;

(3) administration to said subject of tautomycetin or a derivative thereof or a pharmaceutically acceptable salt thereof, and one or more immunosuppressant drugs as indicated above;

(4) administration to said subject of tautomycetin or a derivative thereof or a pharmaceutically acceptable salt thereof, associated to other means that alter T cell regulation and/or thymic induction, and in conjunction with one or more immunosuppressant drugs, e.g. as indicated above;

(5) administration to said subject of tautomycetin or a derivative thereof or a pharmaceutically acceptable salt thereof, and bone marrow;

(6) administration to said subject of tautomycetin or a derivative thereof or a pharmaceutically acceptable salt thereof, and bone marrow, associated to other means that alter T cell regulation and/or thymic induction, and in conjunction with one or more immunosuppressant drugs, e.g. as indicated above;

(7) administration to said subject of tautomycetin or a derivative thereof or a pharmaceutically acceptable salt thereof, and donor bone marrow cells, in conjunction with one or more immunosuppressant drugs, e.g. as indicated above;

(8) administration to said subject of tautomycetin or a derivative thereof or a pharmaceutically acceptable salt thereof, and donor bone marrow cells, associated to other means that alter T cell regulation and/or thymic induction, in conjunction with one or more immunosuppressant drugs, e.g. as indicated above.

The adjunct therapy can be administered before, at the same time or after the administration of tautomycetin or a derivative thereof or a pharmaceutically acceptable salt thereof. Different adjunct therapies can be administered to the recipient at different times or at the same time in relation to the transplant event. Various schedules of immunosuppressant therapies and treatment with tautomycetin or a derivative thereof can be used. For example, immunosuppressant therapy may be started from a few hours up to 7 days prior to transplantation; tautomycetin or a derivative thereof may be administered prior to transplantation or a few days after transplantation, e.g. 1 to 9 days, as a single administration or intermittently, and immunosuppressant therapy may be ended simultaneously or a few days after the tautomycetin treatment. In any of the above sequences, donor bone marrow, if desired, can be administered at approximately the time of the transplant or after.

Because the immunosuppressant can be administered before the tautomycetin or derivative thereof treatment and/or other treatments, the present method can also be used with a patient that has undergone an organ transplant and is on an immunosuppressant regimen. This presents a significant opportunity to reduce or eliminate traditional immunosuppressant therapy and its well documented negative side-effects. Also treatment with immunosuppressants prior to transplantation could be particularly useful in cadaveric transplants.

Utility of tautomycetin or a derivative thereof in tolerance induction, as well as in treating disease and conditions as hereinabove specified, may be demonstrated in animal tests for example in accordance with the methods hereinafter described, as well as in clinic where e.g. the transplanted organ or tissue may be submitted to regular biopsy controls and in case of heart transplant additionally to ultrasound scanning.

1. Rat Heterotopic Heart Transplantation

The strain combination used: Male Lewis (RT¹ haplotype) and BN (RT¹ haplotype). The animals are anaesthetised using inhalational isofluorane. Following heparinisation of the donor rat through the abdominal inferior vena cava with simultaneous exsanguination via the aorta, the chest is opened and the heart rapidly cooled. The aorta is ligated and divided distal to the first branch and the brachiocephalic trunk is divided at the first bifurcation. The left pulmonary artery is ligated and divided and the right side divided but left open. All other vessels are dissected free, ligated and divided and the donor heart is removed into iced saline.

The recipient is prepared by dissection and cross-clamping of the infra-renal abdominal aorta and vena cava. The graft is implanted with end-to-side anastomoses, using 10/0 monofilament suture, between the donor brachiocephalic trunk and the recipient aorta and the donor right pulmonary artery to the recipient vena cava. The clamps are removed, the graft tethered retroabdominally, the abdominal contents washed with warm saline and the animal is closed and allowed to recover under a heating lamp. Graft survival is monitored by daily palpation of the beating donor heart through the abdominal wall. Rejection is considered to be complete when heart beat stops. Increases of graft survival are obtained beyond treatment time in animals treated with tautomycetin or a derivative thereof administered i.v. or s.c. at a daily dose of up to 1 mg/kg for 1-12 weeks, e.g. 4 weeks.

2. Combined Therapy

The above procedure is repeated, except that the animals are additionally treated with one or more immunosuppressant, e.g.:

d-1, d=0 and d+21 with FTY720 at a dose of 0.3 mg/kg/day p.o., and/or 40-O-(2-hydroxyethyl)-rapamycin at a dose of 0.5 mg/kg/day p.o.; or

anti-CD154 antibodies at a dose of 10 mg/kg/day i.v. or i.p. 1-3 times per week either alone or with FTY720 at a dose as indicated above, and/or 40-O-(2-hydroxyethyl)-rapamycin at a dose as indicated above. The treatment may last from 1 up to 12 weeks. Significant increase of graft survival lasting beyond the treatment time is obtained.

For all the above indications the required dosage will of course vary depending upon, for example, the host, the mode of administration and the severity of the condition to be treated. In general, however, satisfactory results are obtained by administration, e.g. i.v., i.p. or i.m., in the order of from 0.01 to 1 mg/kg/day of tautomycetin or a derivative thereof or a pharmaceutically acceptable salt thereof, or at a higher dose in case of p.o. administration. An indicated daily dosage for patients is in the range from about 1 to about 100 mg,

Tautomycetin or a derivative thereof may be administered by any conventional route, for example parenterally e.g. in form of injectable solutions or suspensions, or orally using a conventional absorption enhancer.

Where tautomycetin or a derivative thereof or a pharmaceutically acceptable salt thereof, is administered in conjunction with an immunosuppressant drug, dosages of the immunosuppressant drug will of course vary depending on the type of drug employed, on the specific drug employed, on the condition to be treated, and so forth. The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

As an example, FTY720, e.g. in hydrochloride form, may be administered at a daily dose of 0.1 to 25 mg p.o. The mTOR inhibitor, e.g. rapamycin or 40-O-(2-hydroxyethyl)-rapamycin may be administered at a daily dose of 0.25 to 25 mg p.o., preferably 0.75 to 5 mg p.o. The anti-CD154 antibody and/or any form of CTLA4-Ig fusion protein (or any other costimulation-blocking compound) may be administered at a dose of from 2 mg to 2 g.

In accordance with the foregoing the present invention provides in a yet further aspect:

3. A therapeutic combination, e.g. a kit, comprising a) tautomycetin or a derivative thereof in free form or in pharmaceutically acceptable salt form, and b) at least one second agent selected from an immunosuppressant, immunomodulatory and a costimulation blocking compound as indicated above. Component a) and component b) may be used concomitantly or in sequence.

1. A method for inducing or modulating T or B cell tolerance in a subject in need of such treatment, comprising administering to said subject an effective amount of tautomycetin or a derivative thereof in free form or in pharmaceutically acceptable salt form.

2 A method for inducing apoptosis of activated T cells in a subject in need of such treatment, comprising exposing said subject to an effective amount of tautomycetin or a derivative thereof in free form or in pharmaceutically acceptable salt form.

3 A method for inhibiting a rejection response in a recipient subject by inducing immune tolerance in said subject to foreign mammalian donor cells, tissue or organ, comprising in any sequence the steps of:
a) administering to the recipient an effective amount of tautomycetin or a derivative thereof in free form or in pharmaceutically acceptable salt form;
b) transplanting the donor cells, tissue or organ into the recipient such that a rejection response by the recipient to the donor cells, tissue or organ is inhibited or prevented, and optionally
c) transplanting bone marrow donor cells or other hemapoietic-precursor-cells or lymphoid donor cells to the recipient such that a graft-versus-host response by the donor cells against the recipient is prevented or inhibited.

4 A method for delaying progression of, attenuating severity of, suppressing, mitigating or treating autoimmune disorders or diseases in a subject by inducing immune tolerance in said subject, the autoimmune disorders or diseases being dependent on activation of lymphoid cells, which method comprises administering to said subject an effective amount of tautomycetin or a derivative thereof in free form or in pharmaceutically acceptable salt form.

5 A method for treating malignancies in a subject in need thereof, comprising administering to said subject a therapeutically effective amount of tautomycetin or a derivative thereof in free form or in pharmaceutically acceptable salt form.

6. A method according to claim 1 comprising co-administering, concomittantly or in sequence, at least a second agent which is selected from an immunosuppressant, an immunomodulatory and a costimulation blocking compound.

7. A method according to claim 1 comprising the association with other means that alter T cell regulation and/or thymic induction.

8. A method according to claim 7, comprising co-administering, concomittantly or in sequence, at least a second agent which is selected from an immunosuppressant, an immunomodulatory and a costimulation blocking compound.

9. A method according to claim 1, comprising co-administering bone marrow concomittantly or in sequence.

10. A method according to claim 1, comprising co-administering, concomittantly or in sequence, bone marrow, other means that alter T cell regulation and/or thymic induction, and at least a second agent which is selected from an immunosuppressant, an immunomodulatory and a costimulation blocking compound.

11. A method according to claim 1, comprising co-administering, concomittantly or in sequence, donor bone marrow and at least a second agent which is selected from an immunosuppressant, an immunomodulatory and a costimulation blocking compound.

12. A method according to claim 1, comprising co-administering, concomittantly or in sequence, donor bone marrow and other means that alter T cell regulation and/or thymic induction.

13. A pharmaceutical combination comprising a) tautomycetin or a derivative thereof in free form or in pharmaceutically acceptable salt form and b) at least a second agent which is selected from an immunosuppressant, an immunomodulatory and a costimulation blocking compound.
